(19) United States
(12) Patent Application Publication
(10) Pub. No.: US 2008/0215010 A1
(43) Pub. Date: Sep. 4, 2008

(54) APPARATUS AND METHOD FOR USING AN INTRAOSSEOUS SPACE FOR MOVING FLUID INTO AND OUT OF THE BODY

(76) Inventors: Theodore A. Silver, Central Islip, NY (US); David J. Wood, Glen Cove, NY (US)

Correspondence Address: David Aker, 23 Southern Road, Hartsdale, NY, 10530, US

(21) Appl. No.: 11/941,961

(22) Filed: Nov. 18, 2007

Related U.S. Application Data
(63) Continuation-in-part of application No. 11/068,508, filed on Feb. 28, 2005.
(60) Provisional application No. 60/548,045, filed on Feb. 26, 2004.
(60) Provisional application No. 60/859,775, filed on Nov. 17, 2006.

(51) Int. Cl. A61M 39/00

(52) U.S. Cl. 604/175

(57) ABSTRACT

An apparatus for communication between a site within a body and a source, depot, reservoir, device or machine external to the body via the mouth of the body, comprising an implant for placement in tooth-supporting bone, the implant having a first portion extending into the mouth external to the tooth-supporting bone, a second portion for being embedded in the tooth-supporting bone, and an opening extending along the implant from the first portion to the second portion. A sealing cap containing an antimicrobial substance may be provided to provide a seal when the apparatus is not in use. An external pump may infuse various medications or liquids, including those required by a person having a diabetic condition.

### RELATED APPLICATIONS

This application is a continuation-in part of patent application Ser. No. 11/068,508, which was filed on Feb. 28, 2005, which claims priority from provisional patent application Ser. No. 60/548,045 filed on Feb. 26, 2004. This application also claims priority from provisional application Ser. No. 60/859,775, which was filed on Nov. 17, 2006. The entire contents of all of these applications are incorporated herein by reference, for all purposes.

### BACKGROUND OF THE INVENTION

1. Field of the Invention

The present invention relates to apparatus and methods, for the movement of fluid into and out of the human body. More particularly, it is specifically directed to mediating such movement without traditional cannulae or catheters that pierce the skin and create significant risk of infection.

2. Background Art

Movement of fluid into and/or out of the blood vessels or other spaces deep to the skin is a necessary incident to many therapies including, most commonly, the intravenous administration of medication and electrolytic fluid. Parenteral nutrition/hyperalimentation requires that fluid move into the vascular system from a reservoir outside the body. In the process of renal dialysis, fluid is removed from the body, processed, and returned. Where fluid is to be moved from a source outside the body to blood vessels, machinery, reservoirs or other sites within the body, the prior art resorts to needles, cannulae, and catheters that traverse the skin and so create an opening between the environment and the site to which flow is directed. If the process is to be conducted over a long term, the opening remains in place and presents a risk of serious infection. Modern protocol requires that the ordinary intravenous catheters inserted for fluid and medication infusion (most commonly in a vein at or near the wrist) be changed every three days in order to avoid infection.

Hyperalimentation, chemotherapy, long-term intravenous infusion, and frequent, repeated blood drawing are often facilitated by indwelling catheters called, more specifically, “tunneled central venous catheters” and “peripherally inserted central catheters” (PICCs). Once placed, a tunneled central venous catheter presents access ports at the chest wall. It penetrates the skin of the thoracic wall and then tunnels upward under the skin to reach the superior vena cava. The PICC, once placed, runs through the skin (piercing the skin, sometimes at the upper limb, to enter the brachial vein) and then reaches the subclavian vein (and, perhaps, the superior vena cava). Both tunneled central venous catheters and PICCs have infection as a chief cause of morbidity and mortality.

All processes requiring the long term delivery of fluid to the inside of the body would be rendered safer and more useful by a device that would mediate the delivery without the need for ongoing communication, through the skin, between environment and bodily interior. Prior art offers no such device.

Furthermore, some conditions (diabetes and hemophilia, for example), require not a long term cannula or indwelling catheter but regular and repeated subcutaneous, intramuscular or intravenous injection by needle and syringe. Injection does not raise any extraordinary risk of infection, but it is painful and inconvenient (especially for children). Repeated venupuncture (as is necessary, for example, to renal dialysis and to chronic conditions that require repeated intravenous infusion or blood sampling) damages the peripheral veins; access to usable veins often becomes enormously problematic. Patients whose conditions require, regularly and repeatedly, that fluid be moved between the inside and outside of the body would benefit from a device that mediates the movement without the need for venupuncture or injection. Prior art offers no such device.

The prior art does teach the injection of medication into bone/intraosseous space as an established procedure largely equivalent, in most ways, to intravenous injection. Intraosseous injection is practiced, now primarily in children, for emergency situations in which a vein is not available. On an emergency basis, a needle is passed through the skin, through the surface, usually of the tibia (shin bone) and on into the space within the bone, that being the intraosseous space. Much intraosseous space is, generally, dense with vessels that are continuous/contiguous with the venous circulation, and medication or fluids generally can thus be delivered directly into the space, and achieve a result substantially similar to that of intravenous injection. Furthermore, venous blood can be removed from the intraosseous space via the same route.

A bone often used is the tibia. As its primary disadvantage, it raises the risk of osteomyelitis; a particularly virulent, potentially lethal infection.

### SUMMARY OF THE INVENTION

It is an object and feature of this invention to create on an ongoing or regular/intermittent basis, an avenue for flow of fluid between some source or device outside the human body to some vessel, organ, space, position, system, reservoir or device located inside the body (meaning any site deep to the skin), such to be achieved without piercing the skin and so without creating, for periods long or short, any open communication between skin and outside environment.

It is a further object and feature of this invention to eliminate, substantially, the risk of infection associated with “cannulae,” “peripherally inserted central catheters,” “tunneled central venous catheters,” “indwelling catheters” and related devices now devoted to introducing electricity or fluid to the inside of the human body on long term bases.

It is a further object and feature of this invention to eliminate, substantially, the necessity for regular, repeated needle puncture associated with syringes, intravenous catheters, and related devices now used to facilitate regular, repeated injection of medication or other processes that require regular access to blood vessels or other spaces deep to the skin.

It is a further object and feature of this invention to provide a device that allows for the movement of fluid between the outside of the body and a blood vessel(s) or other site(s) located anywhere deep to the skin, without opening the skin and thereby to (a) reduce or substantially eliminate the pain, inconvenience, vascular damage, inaccessibility and risk associated with repeated injection, piercing of skin, and puncture of blood vessel and, (b) in the case of permanent/long term delivery (or withdrawal) of fluid, substantially eliminate the serious and significant risk of infection now associated with such undertakings.

It is still another object to use tooth implant technology as described herein, and preferably a tooth type implant in the maxillary bone, for intraosseous injection, so that medication can be introduced to adults and children (and blood withdrawn from adults and children) via the intraosseous maxillary space, through a permanently implanted port, while raising no risk of serious infection.

It is a further object of the invention to provide a method and apparatus for keeping the portion of such an implant that extends into the mouth free of bacteria or other microbes, which could cause infection.

These objects and others are achieved in accordance with the invention. As is elaborated in the text and illustrations describing the preferred embodiments, the apparatus in accordance with the invention comprises, in a first tunnel drilled vertically downward through the mandible into which is driven, after the fashion of a screw, a hollow structure, roughly cylindrical in shape, composed of a non-reactive material, threaded on its outer surface similar to that of a dental implant footing/anchor. In a second tunnel of smaller caliber (drilled medially from the mandible's lateral aspect, laterally from the mandible's medial aspect, or upward from its inferior aspect so positioned and directed as to cause the internal ends of the two tunnels to communicate), with the device implanted (as discussed below) the second narrower tunnel (a) creates communication and continuity through the mandible of conductors or piping, while (b) preventing damage to or extrusion of the device by the downward force potentially imposed on the device when the patient bites or chews.

For fluid flow, the vertically oriented sleeve may be fitted with valved tubing, sealed to the sleeve against microbes by dental sealant/cement, and connected outside the body to the relevant source, reservoir, or device. The tubing passes through the larger tunnel, on into the smaller one and thence from the lateral or inferior aspect of the mandible to the blood vessel or other site from which or to which flow is directed coursing, always, beneath the skin.

### BRIEF DESCRIPTION OF THE DRAWINGS

The foregoing aspects and other features of the present invention are explained in the following description, taken in connection with the accompanying drawings, wherein:

FIG. 1 shows a right lateral view of the mandible in sagital section, with a tooth removed and tunnel drilled in the resulting space, in accordance with the invention.

FIG. 2 shows a perspective view of a hollow cylindrical implant in accordance with the invention.

FIG. 3 shows a right lateral view of the mandible in sagital section, with the implant shown in FIG. 2 driven into the tunnel shown in FIG. 1.

FIG. 4 shows a sectioned view of a coaxial conductor driven into the implant shown in FIG. 2, which implant, in turn, is driven into the tunnel shown in FIG. 1.

FIG. 5 shows a top view of the coaxial conductor shown in FIG. 4.

FIG. 6 shows a right lateral view of the mandible in sagital section, with implant and coaxial conductor in place, and with a second tunnel drilled medially from the lateral aspect of the mandible, into which may be inserted an electrical conduit leading to some site within the body.

FIG. 7 shows a right lateral view of the mandible in sagital section with communication between the tunnel shown in FIG. 1 and the second tunnel shown in FIG. 6.

FIG. 8 shows a frontal view of gingival tissue surrounding mandibular tissue, with implant and coaxial conductor in place, and with the second tunnel of FIG. 6 carrying a conduit from the coaxial conductor to some site within the body.

FIG. 9 shows a left lateral view of the inside of the mouth with conductors entering/exiting it via either the oral orifice, a skin tunnel through skin under the lower lip, or a skin tunnel through the cheek, and a “dental electroface” removably attached (in “snap-on-snap-off” fashion) to an artificial tooth.

FIG. 10A shows a sectioned view of a first (vertical) fluid conduit/pipe/tube threaded on its external surface, driven into the implant shown in FIG. 2 which implant, in turn, is driven into the tunnel shown in FIG. 1.

FIG. 10B shows, in perspective, a coupler that may connect the first conduit to a second conduit in accordance with the invention (as shown in FIG. 11).

FIG. 11 shows a frontal view of gingival tissue surrounding mandibular tissue, with implant and first (vertical) fluid conduit/pipe/tube in place, and with the second tunnel shown in FIG. 6 carrying a second (horizontal) fluid conduit/pipe/tube connected at roughly a right angle to the first (vertical) conduit (via the coupling shown in FIG. 10B) that second conduit then to carry fluid (via additional conduits as necessary) to or from some site within the body.

FIG. 12 shows, in schematic fashion, electric current moving from a source outside the body through an electrical conduit that runs into the mouth, connected then to the coaxial insert (not shown in this schematic figure), thence to a second conduit (not shown in this schematic figure) and, ultimately, to an LVAD, all in accordance with the invention.

FIG. 13 is an enlarged, perspective, exploded view of an antiseptic cap to be applied in accordance with an alternate embodiment of the invention.

FIG. 14 is an enlarged cross sectional view of the cap of FIG. 13 being connected to a port in accordance with an alternate embodiment of the invention.

FIG. 15 is an enlarged cross sectional view of the cap of FIG. 13 connected to the port in accordance with the alternate embodiment of the invention.

FIG. 16 is a diagram of a system for administering insulin and/or glucagon in accordance with the invention.

### DETAILED DESCRIPTION OF THE PREFERRED EMBODIMENTS

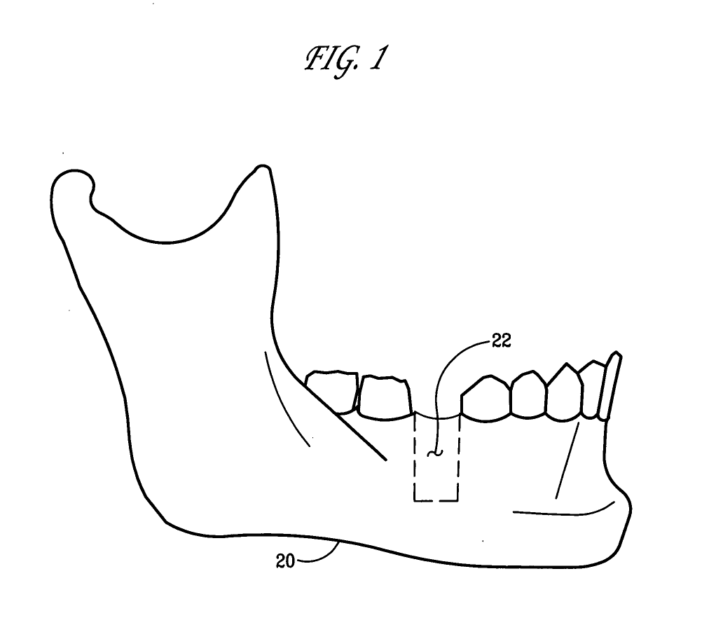

Referring to FIG. 1, a premolar/bicuspid or molar (or other) tooth has been removed from the mandible 20 (on right or left side). In the resulting space there is drilled downward through the mandible a vertically oriented tunnel 22, 10 mm to 15 mm deep and 4 mm to 7.5 mm in diameter (or of such other depth and diameter as suits the purpose described in the next sentence).

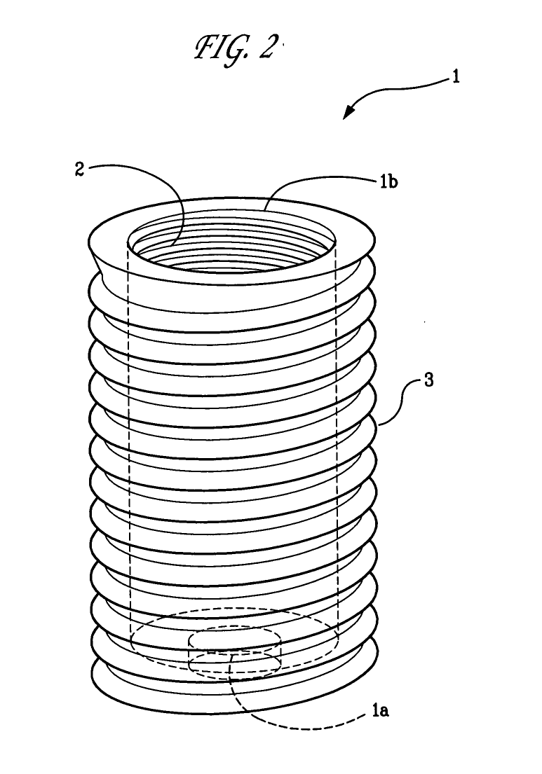

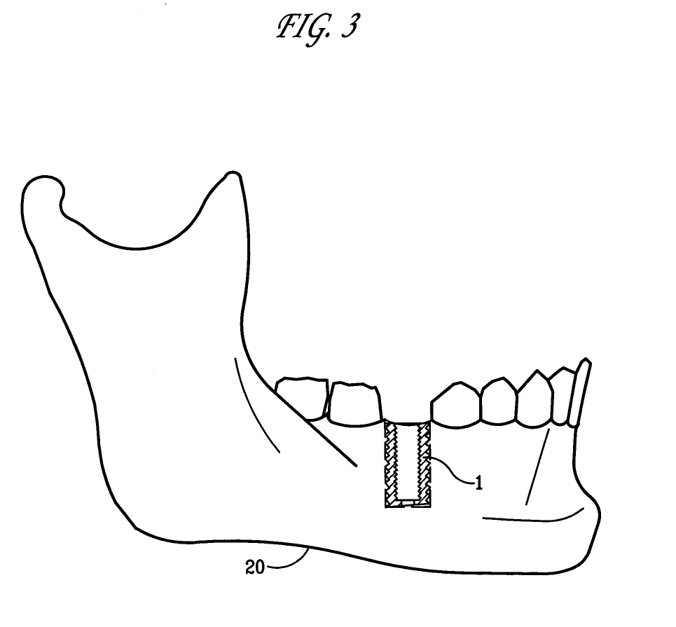

Referring to FIG. 2, driven into that tunnel, after the fashion of a screw, is a hollow entity or screw 1, roughly cylindrical in shape, open at both ends (the lower end 1A having a smaller diameter than the upper end 1B), composed of titanium, platinum or other non-reactive material. Its outer surface is threaded, as shown at 3 to facilitate fixture within the vertical tunnel 22 as shown in FIG. 3, with subsequent bone growth around it creating such a seal between sleeve and bone as does not permit the passage of microbes from the mouth or bodily exterior.

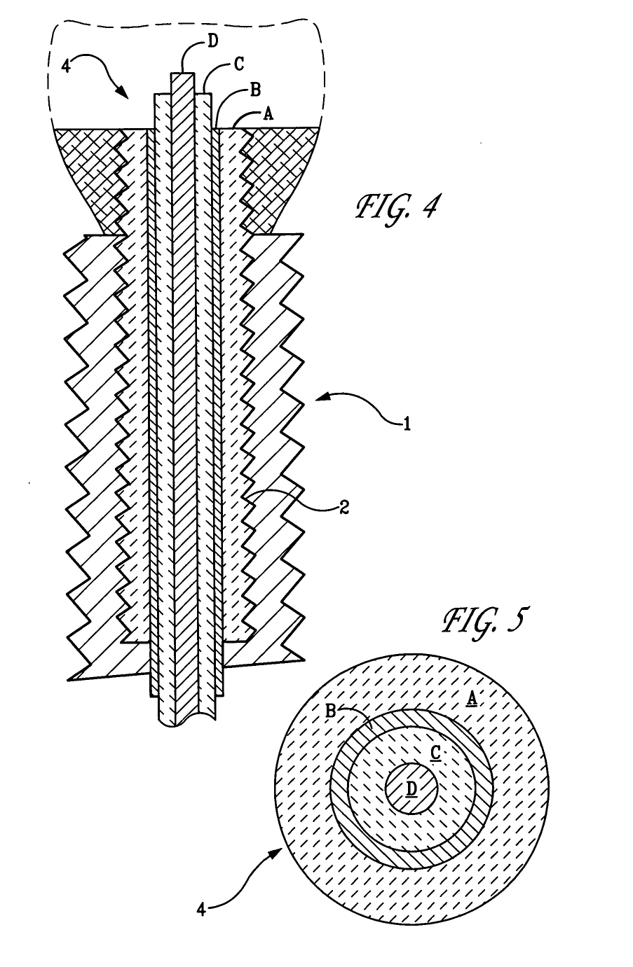

Referring to FIG. 4, the inner hollow aspect of the inserted component or screw 1 is also threaded, as shown at 2 and functions as a sleeve into which is driven, also after the fashion of a screw, a threaded cylindrical coaxial conductor or coaxial insert 4, which together with dental sealant/cement, creates such a seal between coaxial insert and sleeve, as does not permit the passage of microbes from the mouth or bodily exterior.

Referring to FIG. 4 and FIG. 5, the coaxial insert 4 is layered in composition from its external surface inward with a layer A of an insulating or dielectric material (plastic or ceramic for example) threaded, as just described, a conductor layer B, a layer of an insulating or dielectric material C, and a layer or core of a second conductor D.

The coaxial insert 4 protrudes through the sleeve at both its upper end and its lower ends (FIG. 4), thus presenting (a) an upper male coaxial terminus to the mouth and a lower male coaxial terminus to the mandibular interstitium (FIG. 6).

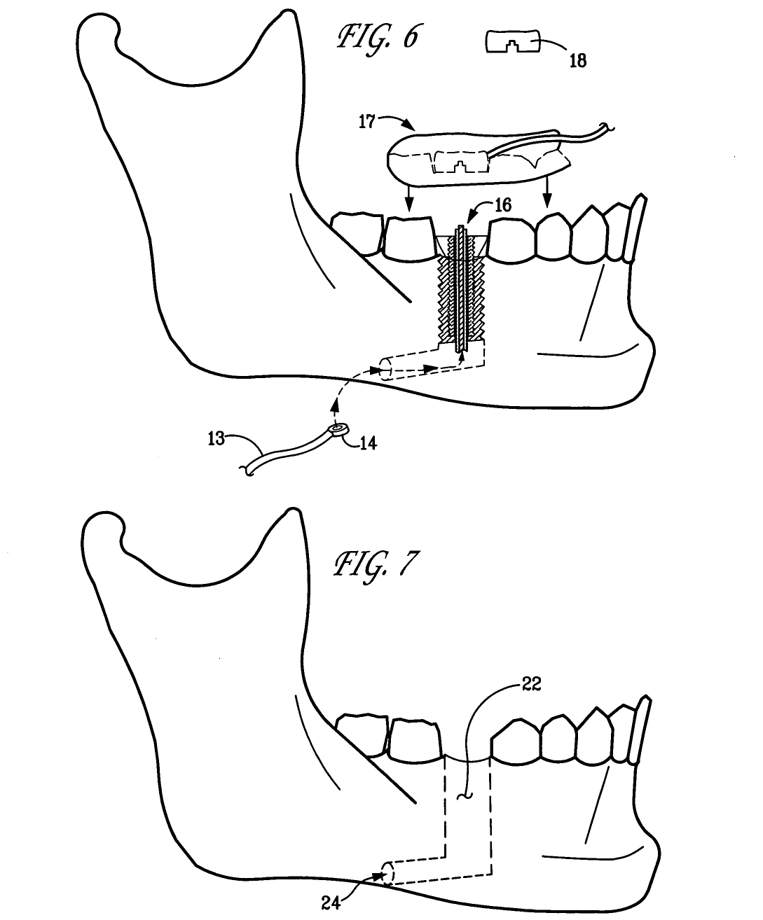

Referring to FIG. 7, a horizontally oriented tunnel 24 is drilled medially through the mandible), with the antero-posterior and infero-superior positions of horizontal and vertical tunnels corresponding sufficiently to create communication between the medial end of the horizontal tunnel 24 and the inferior end of the vertical tunnel 22. Alternatively the tunnel could be drilled laterally from the medial aspect of the mandible to join the vertical tunnel.

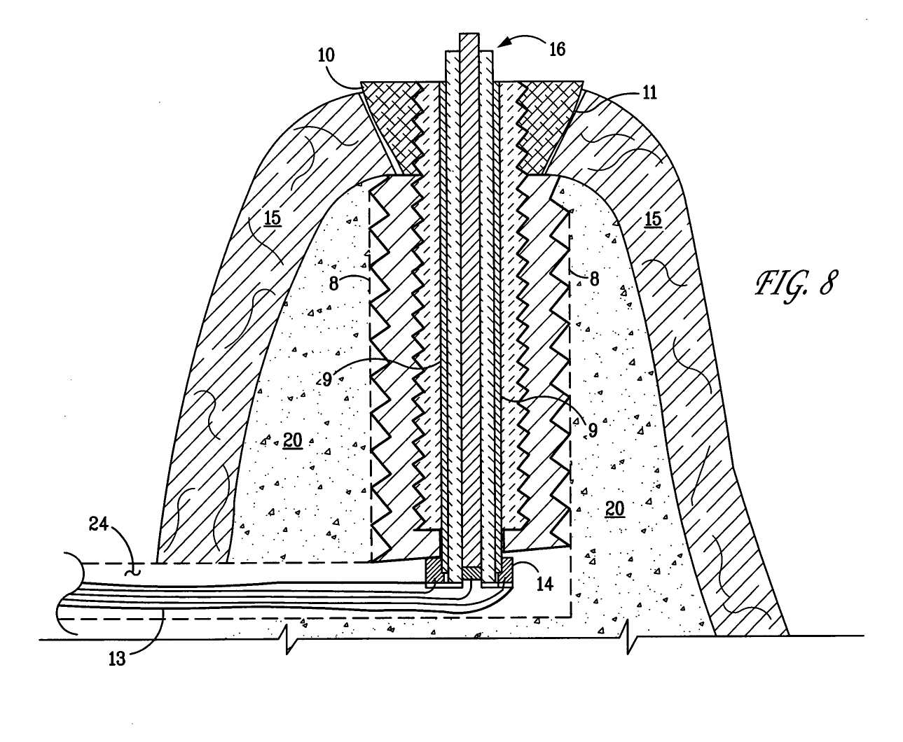

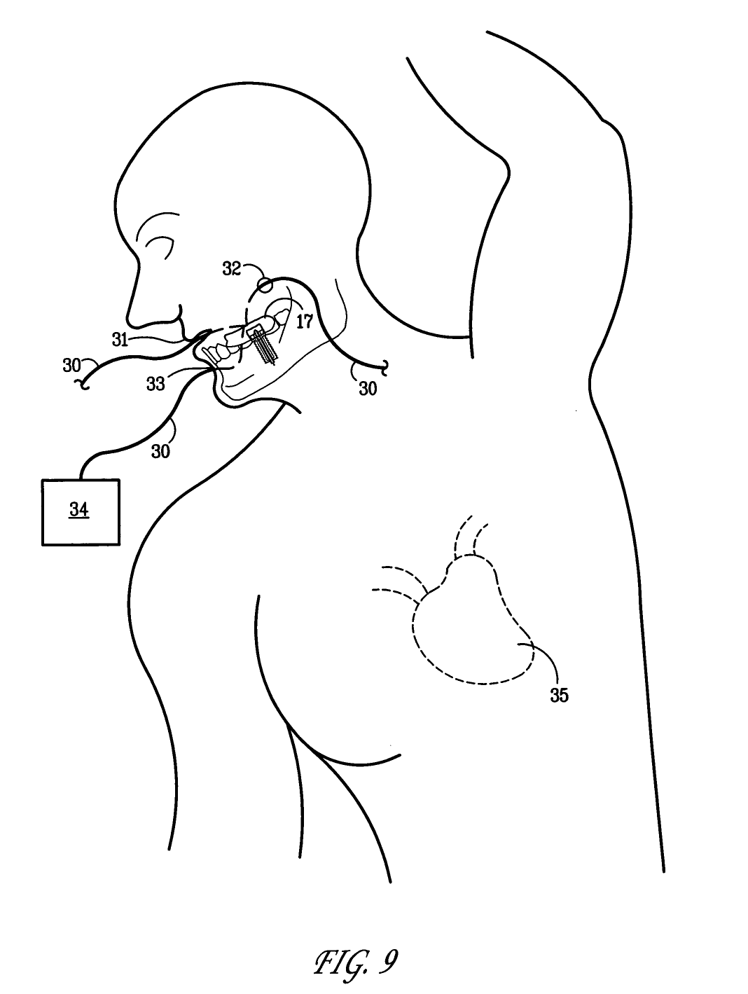

Referring to FIG. 6, in the horizontal tunnel 24 there is placed a second coaxial conductor 13, that presents to the lower coaxial male terminus a female coaxial terminus 14, threaded on its internal surface (roughly after the fashion, for example, of a screw-on bottle top or light bulb socket). Free space between horizontal conductor 13 and horizontal tunnel 24 (FIG. 8) may be filled, as necessary, by application of a substance(s) that replaces bone or stimulates its formation, such as the “putty” or “bone in a bottle” or “bone formation powder” familiar to dentistry.

Referring to FIG. 8, the male terminus at the bottom of insert 4, is fitted into the female terminus 14 by screwing, in watertight fashion. Corresponding to the male terminus, the female terminus is layered in composition from its outermost to innermost aspects with: an insulating or a dielectric material (plastic or ceramic for example) threaded, as just described, to facilitate connection between male and female termini; a first conductor; an insulating or dielectric material, and a second conductor.

The device is installed in two parts through two separate tunnels, a wider one and a narrower one, as just described in order to (a) create communication and continuity through the mandible of the conductive pathway while (b) preventing damage to or extrusion of the device by the downward force potentially imposed on it when the patient bites or chews.

In the mouth, the protruding male terminus of insert 4 may be flared, in or after the fashion of a standard dental implant abutment 10 so to create an antimicrobial seal 11 between coaxial insert and gingival tissue 15, wherefore, all arrangements just described create a single coaxial conductive system extending from the male terminus 16 in the mouth through the upper aspect of the mandible 20, emerging at the lateral aspect of the mandible 20 deep to the skin, with antimicrobial seals between (a) mandible 20 and sleeve at 8, (b) sleeve and coaxial insert 9, and (c) coaxial conductor and gingival tissue at 11.

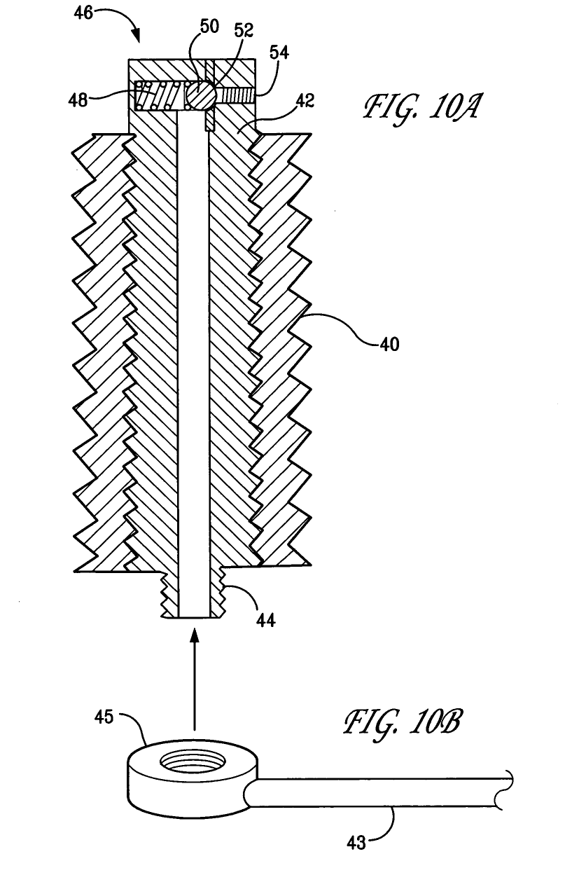

As illustrated in FIG. 6 and in FIG. 9, in the mouth a dental electro-interface 17, which may be a removable (“snap-on-snap-off”) component composed of acrylic (blow molded, or thermal molded, for example), form fitted after the fashion of a prosthetic dental lower partial plate/ridge/retainer, serves as female terminus, and sits securely over (a) several natural teeth (or such prosthetic substitutes as may characterize the patient's dental status), and (b) the upper male coaxial terminus 16, where it presents to the upper male terminus an upper female coaxial terminus. A prosthetic tooth 18 is placed over male terminus 16 when electro-interface 17 is not in use.

From the upper female terminus of electro-interface 17 just described, a coaxial conductor 30 (or other arrangement that embodies (at least) two insulated electrical leads) proceeds out of the mouth via some suitable route which might, for example, run (a) directly through the oral orifice or mouth as shown at 31, or (b) through a skin tunnel (like the “piercings” that accommodate jewelry) traversing the mucosa, muscles, subcutaneous tissues and skin situate between (i) the maxillary and mandibular bones (the buccal/“cheek” area) as shown at 32, or (ii) the lower labia and mental protuberance (between the “lower lip” and “chin”), as shown at 33, all as illustrated in FIG. 9.

The coaxial insert joins a cable (or other arrangement that embodies at least two insulated electric leads), which runs from its exit site at the mouth to a power source (such as a battery) 34, worn outside the body.

At the lateral end of the horizontal tunnel 24, the horizontally oriented coaxial conductor 14 presents a male or female end suitable for connection to a reciprocal connector (or it may present any conductive system that embodies at least two insulated electrical leads/conductors) which, in turn, will extend the conducting system to another internal area of the body, such as for example 35, by coursing, preferably always, deep to the skin.

The embodiment thus facilitates the introduction of electric power from (a) a battery source worn outside the body to an electrical device inside the body, or (b) another current source located outside the body (derived, for example, from a power supply connected to household current) into the body for the purpose of charging/recharging a battery located within the body which, in turn, directly powers an electrical device inside the body, all by a route which, unlike all other methods and devices now directed to that same purpose, leaves no open communication between the outside environment and any space deep to the skin, so that the usual risk of lethal infection is substantially eliminated.

### Fluid Flow

In the preferred embodiment, in the manner described with respect to FIG. 1, FIG. 3, and FIG. 6 above, a premolar/bicuspid or molar (or other) tooth is removed from the mandible (on right or left sides). In the resulting space there is drilled downward through the mandible a vertically oriented tunnel, 10 to 15 mm deep and 4 to 7.5 mm in diameter (or of such other depth and diameter as suits the purpose described in the next sentence). Driven into that tunnel, after the fashion of a screw, is a hollow entity, roughly cylindrical in shape, open at both ends (the lower end having a smaller diameter than the upper end), composed of titanium, platinum or other non-reactive material. Its outer surface is threaded to facilitate fixture within the vertical tunnel as just described, with subsequent bone growth around it creating such a seal between sleeve and bone as does not permit the passage of microbes from the mouth or bodily exterior.

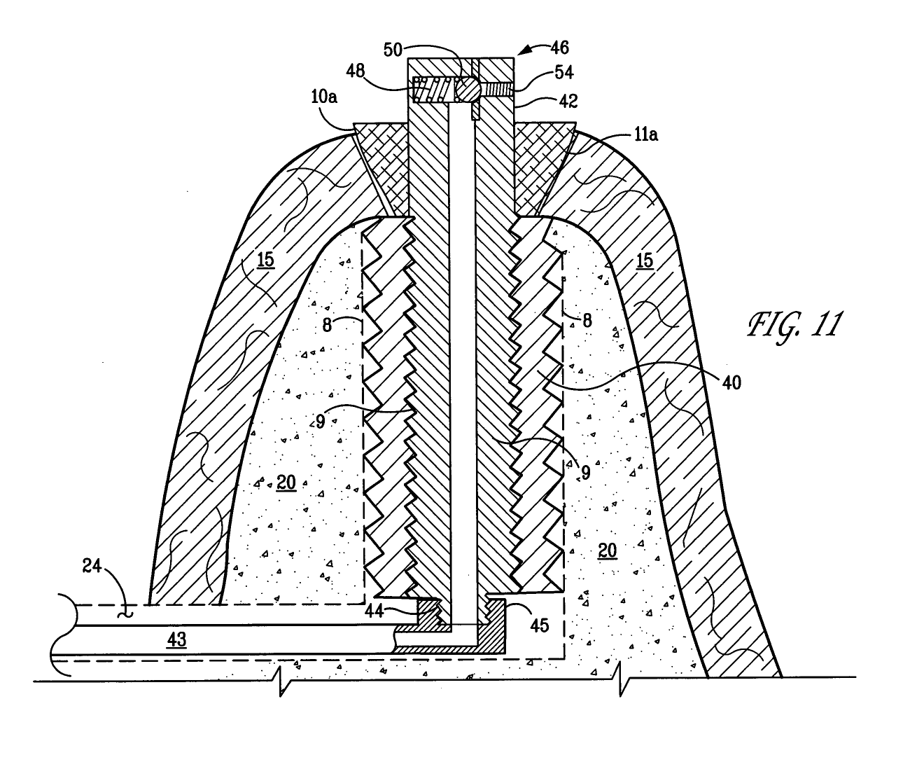

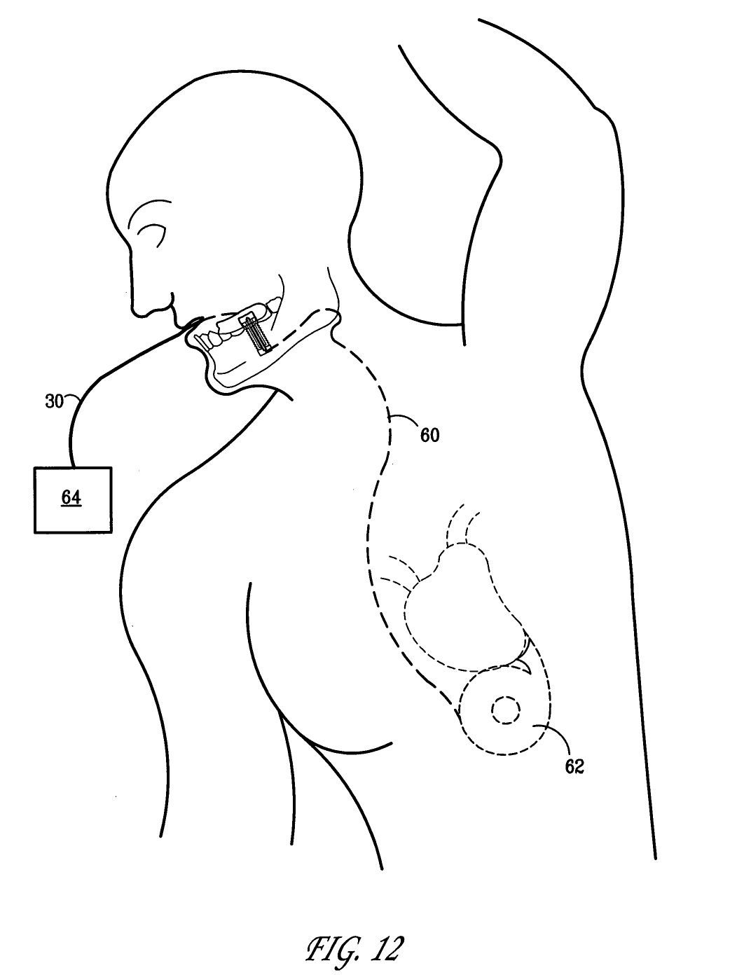

Referring to FIG. 10A, the inner hollow aspect of the inserted component is also threaded and functions as a sleeve 40 into which is driven, also after the fashion of a screw, a cylindrical gold fluid conduit/pipe/tube (pipe 42 hereinafter), threaded on its outer surface, which, together with dental sealant/cement creates such a seal between pipe and sleeve as does not permit the passage of microbes from the mouth or bodily exterior.

Referring also to FIG. 11 (which has many of the same reference numerals as FIG. 6, indicating like components), at its upper end the pipe 42 protrudes above the level of the mandible and sleeve 40 and is flared, after the fashion of a standard dental implant abutment 10A so to create an antimicrobial seal 11A between pipe 42 and gingival tissue 15. At its lower end the pipe 42 narrows in caliber so to create a downward projecting threaded nipple 44 protruding through the sleeve 40.

A horizontally oriented tunnel 24 is drilled medially through the mandible, with the antero-posterior and infero-superior positions of horizontal and vertical tunnels corresponding sufficiently to create communication between the medial end of the horizontal tunnel and the inferior end of the vertical tunnel 24. (Alternatively, the tunnel might be drilled laterally from the medial aspect of the right side of the mandible to join the vertical tunnel.) In the horizontal tunnel 24 there is placed a second pipe 43 made of platinum, gold, or other hard non-reactive substance forming, roughly, a right angle with pipe 42 (at a female coupling 45) at the medial end of the horizontal tunnel 24, where the horizontal tunnel 24 communicates with the vertical tunnel 22.

At that same point, the upward pointing end of the “elbow” receives the lower end of the vertical pipe by screwing, so that a continuous watertight system of piping/tubing runs from the upper surface of the mandible into the mandibular interstitium and then, laterally to the mandible's lateral aspect. Free space between the horizontal second pipe 43 and horizontal tunnel 24 is filled, as necessary, by application of a substance(s) that replaces bone or stimulates its formation, such as the “putty” or “bone in a bottle” or “bone formation powder” familiar to dentistry.

The apparatus is installed in two parts through two separate tunnels, a wider one and a narrower one in order to (a) create communication and continuity through the mandible of piping while (b) preventing damage to or extrusion of the device by the downward force potentially imposed on it when the patient bites or chews.)

In the mouth, there is screwed to the upper end of the gold tube or pipe 42 a one-way valve system 46 permitting flow in the inward direction only. Valve system 46 may have a one-way check valve having a spring 48, a ball 50 and a concave wall 52 against which ball 50 rests to form a seal. An opening 54 may have internal threads to receive a threaded needle catheter (not shown). (Alternatively the tube is not open at its upper end, but rather carries a one-way valve as its upper end. Alternatively, the entire tubing system might be bi-luminal (“double barreled) each lumen equipped at its upper end with a valve, one permitting inward flow only and the other permitting outward flow only. Alternatively, for an application such as renal dialysis, in which fluid must move into and out of the vascular system, the patient might be fitted with two separate piping systems, one entering the mandible at each side of the mouth, one to carry fluid inward to the vascular system and one to carry it outward therefrom.

When flow is not to be initiated, the upper end of the pipe is covered by a form fitted (“snap-on-snap-off”) prosthetic tooth. When flow is to be initiated, the prosthetic tooth is removed. Suitable piping or tubing is connected to the valve and the valve is opened. The tubing will exit the mouth (in a manner analogous to that which is shown in FIG. 9, (a) directly through the oral orifice, or (b) through a skin tunnel (like the “piercings” that accommodate jewelry) traversing the mucosa, muscles, subcutaneous tissues and skin situate between (i) the maxillary and mandibular bones (the buccal/“cheek” area) or (ii) the lower labia and mental protuberance (between the “lower lip” and “chin”).

At the lateral end of the horizontal tunnel, the horizontally oriented tube is connected to additional piping/tubing which, in turn, will extend the conducting system to the vessel, space, site or device to which or from which fluid is to move coursing, preferably always, deep to the skin.

The embodiment thus facilitates the movement of fluid into and/or out of the body from or to some source, depot, reservoir or device outside the human body to some site, space, vessel, or device located inside the body all by a route which, unlike all other methods and devices now directed to that same purpose, leaves no open communication between the outside environment and any space deep to the skin, so that the pain, inconvenience, inaccessibility, and risk of lethal infection are substantially eliminated.

### Preferred Applications

### Electrical Conduction

A. As described above with respect to the prior art, Left ventricular devices (LVADs), (bearing the brand names, for example “Heartmate,” “Arrow LionHeart,” and “Debakey,”) require external “drive lines” to deliver power from an external battery pack. (Thoratec, the manufacturer of “Heartmate” has also manufactured a device driven pneumatically so that the abdomen is pierced not by electrical conductors but rather by hoses delivering pneumatic power which, in turn, is generated by electric machinery located outside the body). The electrical drive lines create a grave risk of infection (some 40% of patients dying of infection), this being the LVAD's chief drawback and disadvantage.

The Jarvic 2000 LVAD seeks to address the problem by directing current not through an abdominal piercing but through the scalp (whose vascularity is said to decrease the risk of infection). Undergoing testing in Europe (but not in the U.S.), the Arrow LionHeart LVAD seeks to solve the drive-line infection problem by operating on transcutaneous energy transfer/induction (TET). Shortcomings are likely to include (a) malfunction through electromagnetic interference, and (b) limitation as to the magnitude of power that can be introduced.

Referring to FIG. 12, in accordance with the present invention, in one preferred application of the preferred embodiment, conductors 60 emanating from the lateral aspect of the mandible (or from the medial aspect if the horizontal tunnel is drilled laterally from the medial aspect of the mandible toward the vertical tunnel) are tunneled under the skin and connected to the LVAD 62, in place of the drive-lines offered by prior art. Conductors emanating from the mouth (as described in the preferred embodiment) lead to a battery 64 (worn on the belt or in a pocket, for example), and the battery 64 powers the LVAD. (The LVAD is also equipped with an internal battery). Circuitry allows for current to run directly from the external battery to the LVAD and also for recharging of the internal battery from some other source (derived, for example, from household current). If, for some reason, the patient wished temporarily to be free of the external wiring, she would remove the dental electro-face, replace it with an artificial tooth, disconnect the wiring from the dental electro-interface, and remove both wiring and dental electro-interface from her mouth. The LVAD would then run on its internal battery, which would be subject to recharging when the connections were restored.

B. For any person who has lost the rectum (usually with some length of colon as well) and is thus consigned permanently to a colostomy bag, a second preferred application of the preferred embodiment powers a one-way peristaltic pump fixed to a synthetic colon that is drawn distally to an artificial (or residual/natural) anus located at or near the perineum. The synthetic colon, in turn, is bound by an airtight/watertight surgical connection proximally to the distal end of the disrupted colon, and distally to the artificial anus (or residual rectum if there be any that is for some reason non-attachable to the colon). The synthetic colon and peristaltic pump are equipped with a one-way valve system, with pump and valve system being electrically operated from outside the body, thus allowing the patient to control defecation and to eliminate quasi-normally through an artificial (or residual/natural) anal opening. A gauge/alarm/signal system (also electrically operated) alerts the patient to the status of filling at the synthetic colon and thus apprises him as to an appropriate time for elimination. (The synthetic colon will be readily accessible from the outside for cleaning by a “bidet” or similar device.)

In accordance with the preferred embodiment, conductors 60 emanating from the lateral aspect of the mandible (or from the medial aspect if the horizontal tunnel is drilled laterally from the medial aspect of the mandible toward the vertical tunnel) are tunneled under the skin toward the abdomen, through the abdominal subcutaneous tissues, through the abdominal muscles, into the abdominal cavity, and thus connected to the peristaltic pump, valve system, and gauge/alarm/signal system. Conductors 30 emanating from the mouth (as described in the preferred embodiment) lead to a battery (worn on the belt or in a pocket, for example), which powers the peristaltic pump, valve system, and gauge/alarm/signal system. The pump might also be equipped with an internal battery.

Circuitry allows current to run directly from an external battery to the pump, valve system, gauge/alarm/signal system and, also, for recharging of the internal battery from some other source (derived, for example, from household current). If, for some reason, the patient wished temporarily to be free of the external wiring, she would remove the dental electro-interface, replaces it with an artificial tooth, disconnect the wiring from the dental electro-interface and remove both wiring and dental electro-interface from her mouth. The pump would then run on its internal battery, which would be subject to recharging when the connections were restored.

Circuitry leading from the peristaltic pump to the outside of the body can include a switch or switches (carried on the belt or in a pocket, for example). With the circuit closed (switch(es) “turned on”) the valve opens and the pump operates thus facilitating quasi-normal defecation through natural or artificial anal opening.

Surgical connection between the synthetic colon and the living tissue of the true colon may be accomplished by turning an external cuff in the colon and an internal cuff in the synthetic colon (or vice-versa) which at its proximal end would be composed of gold mesh. The internal cuff would then lock into the external cuff. Suturing the two aspects of the colonic cuff together with the gold mesh in between would then cause healing and sealing between the two aspects of the cuff, creating a continuous watertight/airtight channel of colon and synthetic colon. An analogous procedure would be performed at the rectum/anus, thus to create a continuous airtight/watertight canal from colon to anus, via synthetic colon equipped with a peristaltic pump.)

### Fluid Flow

A. Persons whose kidneys malfunction or fail to function at all are treated regularly and repeatedly (several times per week) with the processes of hemodialysis (or, in some cases, peritoneal dialysis). Hemodialysis requires that fluid move outward from the patient's vascular system, then to the relevant machinery, and back to the vascular system. In order to facilitate the inward and outward flow, the patient is fitted with either (a) a bi-luminal (“double barreled”) piping system, each lumen equipped at its upper end with a valve, one permitting inward flow only and the other permitting outward flow only, or (b) two separate piping systems (one entering the mandible at each side of the mouth, for example), one to carry fluid inward to the vascular system and one to carry it outward.
B. Persons whose condition prevents them from feeding via the gastrointestinal tract are treated with parenteral nutrition/hyperalimentation which requires permanent or long term access to a large vein (such as the subclavian vein). In such a patient the pipe emanating from the lateral end of the mandible (as described in preferred embodiment B) is led by additional watertight/airtight coupling to a large vein such as the subclavian. Nutritional fluid/substance flows from its reservoir outside the body through the piping system into the vein. Any outward flow of fluid that the process might necessitate is facilitated either by (a) a biluminal (“double barreled”) piping system, each lumen equipped at its upper end with a valve, one permitting inward flow only and the other permitting outward flow only, or, as described above, (b) two separate piping systems (one entering the mandible at each side of the mouth, for example), one to carry fluid inward to the vascular system and one to carry it outward.

### Injecting Fluid Into an Intraosseous Space

An application for the invention is the infusion of medication (for example insulin) and withdrawal of blood (to facilitate, for example, blood glucose monitoring in diabetics) via an implant in the maxillary bone, essentially as described above, except that the implant's internal end does not lead to any tubing or conduits that then join a blood vessel remote from the tooth. Rather, the internal portion of the implant just reaches and terminates at the intraosseous space of the maxillary bone, so that fluid (medication or other) passes through the implant into the intraosseous space and thence to the venous vessels running through the space so to take up presence within the venous circulation. The implant may be a single implant, as exemplified by screw 1 of FIG. 3 above, installed in tooth supporting bone as described above with respect to FIG. 1, FIG. 2 and FIG. 3 and extending into, and no further than the intraosseous space.

When the device is so structured and designed as to mediate the delivery or removal of fluid to or from the body (be it medication, blood or other fluid), then during periods of nonuse the lumen that results from the pipe or hollow sleeve or screw 1 is to be maintained substantially filled with heparin or other anti-coagulant (in possible combination with a suitable anti-microbial substance), so to oppose the formation of blood clots within said lumen during such period of nonuse.

It is noted that when the device is so structured and designed as to mediate the delivery or removal of fluid to or from a site within the body, including intraosseous space (be it medication, blood or other fluid) then during periods of nonuse the lumen that results from the pipe or hollow sleeve or screw 1 is to be maintained at the end or “tip” of the portion in the mouth, in a closed condition so to prevent the entry or passage of potentially damaging or toxic entities and, further, must be so closed as to maintain sterility and antisepsis so to prevent the entry or passage, specifically of microbes.

Pursuant to one embodiment, design, and mode of operation the “pipe” or screw 1 is equipped at its proximal end (its lower end where the tooth supporting bone is the maxilla and its upper end where the tooth supporting bone is the mandible), with a mechanically actuated gate valve operating, substantially, on the mechanical principles and phenomena associated with an ordinary automobile tire valve. Positive hydraulic pressure applied via a syringe or similar device containing heparin or other suitable anticoagulants (possibly combined with an antimicrobial substance) causes the valve to open and admit the fluid into the pipe's lumen, such fluid ideally and generally to be infused in and only in such volume as will fill the pipe, the pressure and syringe (or other similar device) then to be withdrawn, causing the valve, therefore, to close and leaving the pipe filled with heparin or heparin and the antimicrobial substance, such fluid being immobilized within the pipe because of the valve's closure, so to operate after the fashion of what modern medicine terms a “heparin lock.

In another method, embodiment, design, and mode of operation regarding the filling of the lumen, during periods of nonuse, with heparin or other suitable anti-coagulant (possibly together with a suitable antimicrobial substance), such embodiment, design, and mode of operation calls for two separate devices of the kind described or one such device divided into two chambers or “barrels” so to function separately as though they constituted two separate devices.

One such device or “barrel” is equipped with two one-way ball valves or “check valves” one valve near the end of the first portion (the end protruding within the mouth), and another near the second portion (the end implanted within the tooth-supporting bone), each such valve when maintained in a closed position, to prevent the passage of fluid and, concomitantly, with both in their open positions, the two valves together to permit the passage of fluid as demonstrated in FIG. 1, wherein the two valves are ball valves maintained in a closed position by the action of springs on their proximal sides, each spring capable of such force as to maintain, in a closed position, the ball valve with which it is associated and each spring susceptible to compression by hydraulic pressure which compression will, in turn, move the balls in the direction away from the opening of the mouth (upward if the bone at issue is the maxilla, and downward if the bone at issue is the mandible) so as to permit movement of fluid in a direction that runs from the mouth toward the bone at issue (upward if the bone at issue is the maxilla one, and downward if the bone at issue is the mandible).

The other such device or “barrel” is reciprocally arranged with springs and balls so situated and opposed as to prevent movement of fluid outward when the valves are closed and permit movement of fluid outward when the valves are open.

In the embodiment, design, and mode of operation just described, involving two separate devices or two separate “barrels” and, consequently two separate pipes, the separation being achieved either by implanting separate devices in each of two separate locations within tooth supporting bone, or by so structuring the device as to supply it with two parallel non-connected chambers or barrels, each is filled with heparin anticoagulant (possibly combined with a suitable antimicrobial substance) during periods of nonuse.

In the case of that device or chamber/barrel/pipe, whose open valves permit the passage of fluid inward, the heparin or heparin/antimicrobial combination is infused into the pipe or screw 1, through a syringe or similar device, via hydraulic pressure sufficient to compress the previously mentioned springs so as to open each of the check valves, such quantity of heparin (or other suitable anti-coagulant) in such quantity, ideally and generally no less and no more, as will fill the full volume of the pipe whereafter with cessation and withdrawal of the infusion and hence the removal of the inward directed pressure, the two check valves will close, thus leaving the pipe filled with the infused fluid, such fluid being immobilized within the pipe because of the valve's closure, so to operate after the fashion of what modern medicine terms a “heparin lock”.

In the case of that device or chamber/barrel whose open valves permit the passage of fluid outward (so for example, to mediate the withdrawal of blood), the springs associated with each valve are so designed as to be openable only with a negative pressure substantially greater than the pressure exerted at the pipe's internal end (the end implanted within the bone). After withdrawal of fluid is complete, but while there is still in place such degree of the negative/outward directed pressure as facilitated the withdrawal, sufficient to maintain the valves in an open position, the heparin/antimicrobial combination is infused into the “pipe” (at its upper end in the case of mandibular implantation and its lower end in the case of maxillary implantation) with such pressure as is not sufficient to close the valves and in such volume no more and no less as will fill the full volume of the pipe, whereupon with cessation and withdrawal of the infusion and hence the removal of the outward directed pressure, the two check valves will close, thus leaving the pipe filled with the infused fluid, such fluid being immobilized within the pipe because of the valve's closure, so to operate after the fashion of a heparin lock.

Closure, Sterility and Antisepsis During Nonuse at That End of the Pipe Protruding Into the Mouth

After the device is used to mediate the infusion or removal of fluid (medication, blood, or other), it is readied for a period of nonuse. The pipe or screw 1, filled with heparin other suitable anticoagulant (possibly together with a suitable anti-microbial substance), is sealed at its end within the mouth so as to prevent the entry of any toxic or otherwise damaging entities including, notably, the entry of microbes.

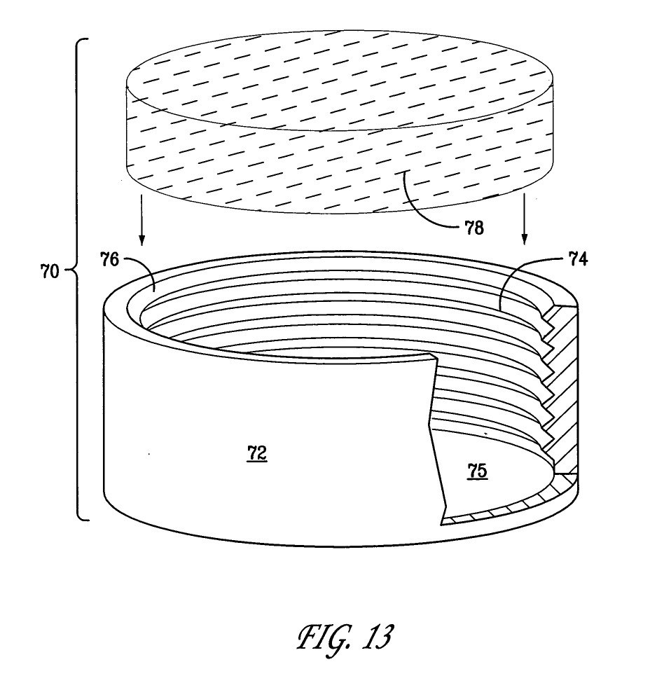

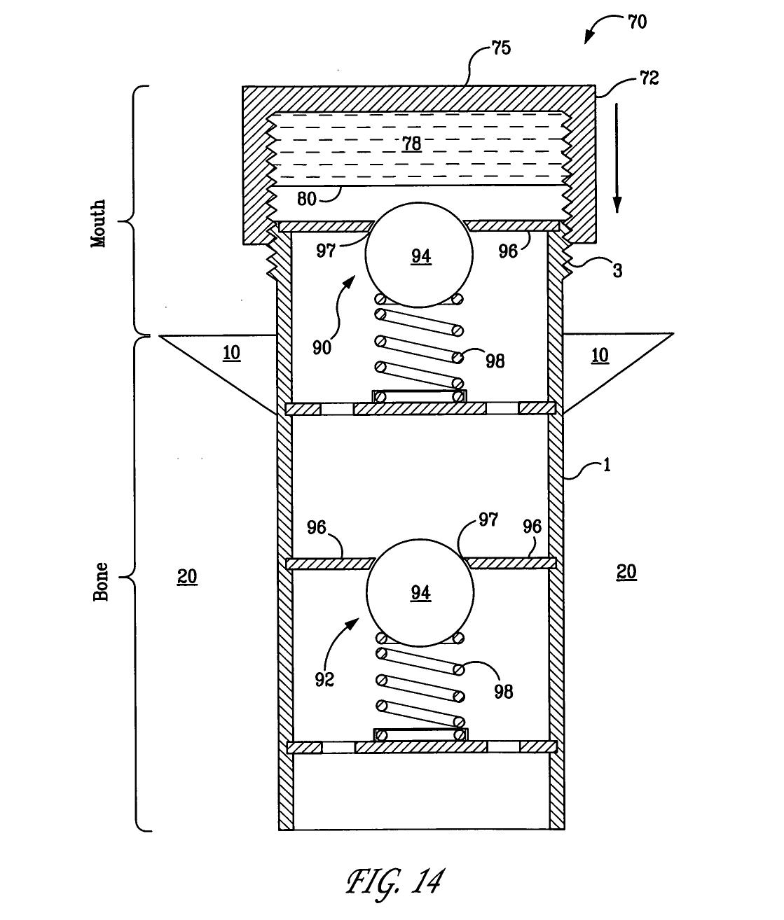

Referring to FIG. 13, a cap shown generally as 70 is shown in an exploded view. Cap 70 is shown in FIG. 13 in isolation, and not attached to any element protruding into the mouth. Cap 70 has a cylindrical cap housing 72 having internal threads 74 which mate with external threads on the portion of the pipe or screw 1 embedded in tooth supporting bone. The housing 72 is closed at one end by an end cap 75, serving as a closed bottom portion, which is an integral part, and formed with cap housing 72. Cap housing 72 is open at the other end 76, and threaded on its internal surface in such size and dimension and nature as to fit, by apposing its thread over the portion of pipe or screw 1 in the mouth (analogous to the screwing of a bottle top onto a bottle).

The cap housing 72 is partially filled from its closed surface toward its open surface with an antiseptic 78 in liquid or gel phase, the degree of filling to correspond to that volume of liquid or gel (as the case may be, and ideally and generally no more or less) as is necessary to surround the whole of that portion of the pipe or screw 1 that protrudes into the mouth, when the cap is applied/screwed onto to such portion as described below.

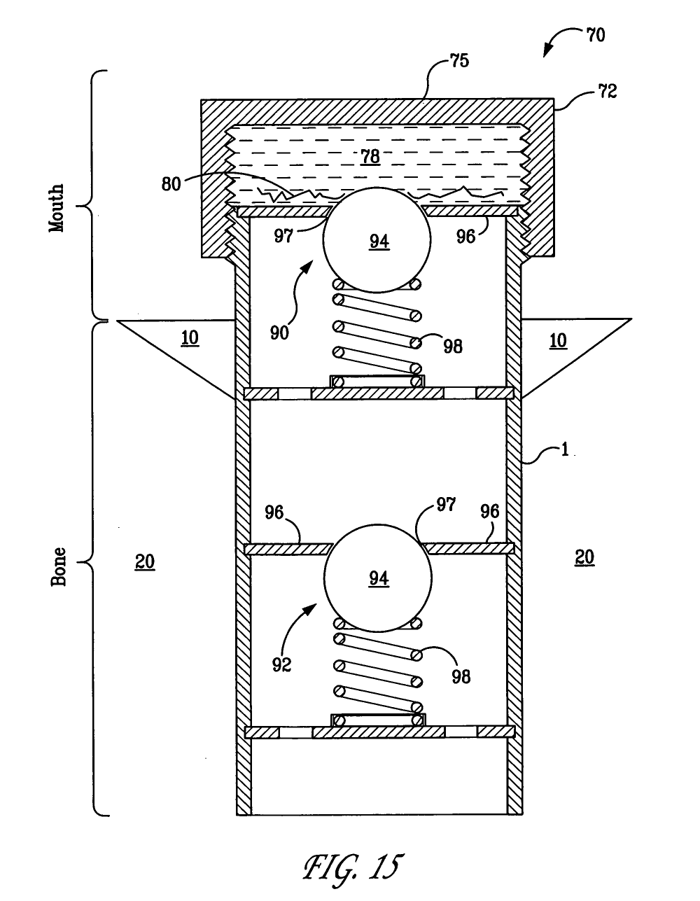

Referring to FIG. 14 (which shows cap 70 in an orientation roughly inverted from that of FIG. 13), at the surface of the antiseptic 78 in gel or liquid phase, is a diaphragm 80 fashioned of cellophane, rubber, micrometallic foil (so-called “aluminum foil”, for example) that fully crosses and covers the inner diameter and circumference of the cap housing 72 so to seal the antiseptic 78 between the diaphragm 80 and the end cap 75.

In FIG. 14, the pipe or screw 1 that protrudes into the mouth threaded on its outer surface is structured with outer threads 3 to receive the cap housing 72 via the simple process of apposing the cap and sleeve, engaging the corresponding threads and screwing on the cap. In FIG. 14, the diaphragm 80 remains whole, having not yet been penetrated by the top of pipe or screw 1.

Referring to FIG. 15, the portion of pipe or screw 1 protruding into the mouth is of such length as to cause its end, as the cap 70 is screwed over it, to puncture the diaphragm 80, so that as the cap 70 continues further to be screwed onto and around the top portion of screw 1, the antiseptic 78, freed from the diaphragm 80 that had confined it, under the prevailing pressure created by the filling of the open space of cap housing 72 containing antiseptic 78, antiseptic 78 begins to flood and fill the threads 74 situated on both the cap housing 72 and the threads at 3 of screw 1, as the two engage.

The depth of the cap housing 72, and the length of the first portion of screw 1 protruding into the mouth over the gum, will be of approximately equal length so that the closed aspect of the cap will reach the proximal end of the first portion and, simultaneously, the top of the cap will reach the abutment earlier referred to, that being the place and entity at which no further upward movement is possible along the outer aspect of the implanted screw 1, so that, at such point in the process the inner surface of cap housing 72 and the outer aspect of the portion of screw 1 protruding into the mouth, each engaged and bound to the other by apposing threads, are flooded and filled with antiseptic 78, the entire structure being closed and sealed at its most protruding end (the upper end in the case of mandibular implantation and its lower end in the case of maxillary implantation) by the end cap 75 integrated with cap housing 72.

As shown in FIG. 14 and FIG. 15, pipe or screw 1 (in bone 20 at one end, and in the mouth at the other end, and having abutment 10 for sealing to the gum) has internal valves shown generally as 90 and 92, for infusion or removal of fluid, as described in detail above, for interchange with the intraosseous space of the maxillary or mandibular bone. Valves 90 and 92 may be of the type having respective balls 94, and members 96 with openings 97 for receiving balls 94 so as to close opening 97, and springs 98 for urging balls 94 against members 96.

Insulin and/or Glucagon Pump

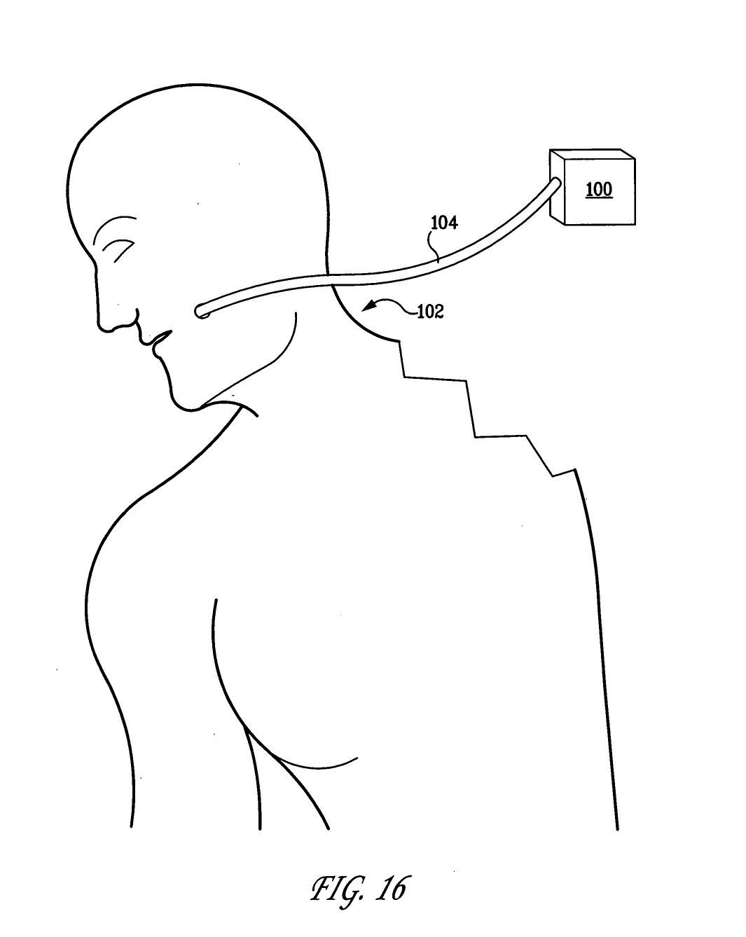

Referring to FIG. 16, the principles of the invention described above may be used to supply a diabetic patient with insulin and/or glucagon or other substances that oppose hyperglycemia and hypoglycemia, respectively. An available automatic apparatus (such as the Paradigm 522 or 722 Real-Time, combination insulin pump and continuous glucose sensor, a device made by Medtronic MiniMed (Medtronic Diabetes of Northridge, Calif.) has a replaceable sensor, implanted under the skin of the side or abdomen of a patient, and helps direct a patient in the operation of an insulin pump.

In accordance with the present invention, a monitor of this kind is used to measure blood glucose level. However, a combination of a monitor and a pump 100, worn outside the body (for example, on a belt or in a shirt pocket) may, without needle puncture or skin opening supply a diabetic patient with insulin and/or glucagon or other substances that oppose hyperglycemia and hypoglycemia, respectively, to be infused into the patient 102 in accordance with the present invention for interchange with the intraosseous space of the maxillary or mandibular bones, via a tube or tubes 104 extending into the mouth, as previously described. Such apparatus may provide for the automatic infusion of glucagon (or other anti-hypoglycemic medication) alone, based on the measured blood glucose level, and may in certain cases serve to save the life of a patient, should the patient be facing the possibility of insulin shock due to a dangerously low blood glucose level. This process may be automated, because if by malfunction, an excess or unnecessary dose of glucagon is infused, such will create no acute danger to the patient's health, but rather will precipitate at worst, a hyperglycemic episode, which posses no acute danger or threat to health or, even symptoms or discomfort. The patient would be unaware of such an episode except via the examination of a blood glucose monitor.

It is also possible to use an implant, pipe or screw for one tooth positioned to monitor blood glucose level, and one at another position to inject insulin and/or glucagon.

Referring to FIGS. 6-12 and to the embodiments specifications and disclosures depicted and described in association therewith, it should be noted that if the device is used to move fluid to or from a blood vessel to some site other than the intraosseous space itself, then, during periods of nonuse, the conducting tubes will be left substantially filled with heparin or other suitable anticoagulant (possibly combined with an antimicrobial substance) after the fashion of what modern medicine terms a “heparin lock” as already described herein, to with, that via the valvular mechanisms and pressure dynamics already described in relation to the fashioning of a “heparin lock”—like entity, the entire length of conducting tube from the mouth to such vessel or vascular structure within the body to which it is ultimately directed, will house an immobilized volume of heparin (possibly combined with an antimicrobial substance) so to prevent unwanted coagulation within the fluid conducting structures.

Referring to all of the embodiments described herein, it should be noted that when the device is so structured as to mediate the delivery or removal of fluid to or from any site within the body, be the fluid medication blood or other, and be the site the intraosseous space or any other site within the body, the lumenal entity/pipe implanted within the tooth supporting bone might or might not, via threads in place on its inner surface be equipped with an inner core/inner pipe, itself threaded on its outside surface that will screw into the implanted pipe or screw and be removable therefrom by the process of unscrewing should it, at any time, be desirable to clean, repair, maintain, or replace it.

It should be understood that the foregoing description is only illustrative of the invention. Various alternatives and modifications can be devised by those skilled in the art without departing from the invention. Accordingly, the present invention is intended to embrace all such alternatives, modifications and variances that fall within the scope of the appended claims.

What is claimed is:

1. An apparatus for communication between a site within a body deep to the skin and a device external to the body via the mouth of said body, said apparatus comprising:
an implant for placement in tooth-supporting bone, said implant having a first portion extending into the mouth external to said tooth-supporting bone, a second portion for being embedded in said tooth-supporting bone and communicating with an intraosseous space of the tooth supporting bone, and an opening extending along said implant from said first portion to said second portion.

2. The apparatus of claim 1, further comprising a one-way valve associated with said implant so as to allow fluid to flow in only one direction through said opening and along said conduit.

3. The apparatus of claim 1, further comprising: a member for being received within the mouth so as to couple to said first portion of said implant, said member having a passageway for coupling to said opening.

4. The apparatus of claim 3, further comprising means for releaseably coupling said member to said first portion of said implant.

5. The apparatus of claim 3, further comprising a conduit for connecting said device to said member.

6. The apparatus of claim 5, in combination with said device.

7. The apparatus of claim 6, wherein said device is selected from the group consisting of a source, depot, reservoir, machine, infusion device and a blood sampling device.

8. The apparatus of claim 1, further comprising blood conducting tubing and/or conductors that extend between said implant and a machine or device being capable of detecting, monitoring, registering and/or recording the glucose concentration of the blood housed within the intraosseous space or the blood vessels contained within said space.

9. The apparatus of claim 8, in which the machine or device is an insulin pump or other machine or device, worn and maintained outside the body, capable of delivering insulin from said pump, machine, or device to the intraosseous space and/or the blood contained within said space and/or the blood contained within the blood vessels within said intraosseous space, via tubing and/or conductors that extend to said insulin pump or other machine or device.

10. The apparatus of claim 8, wherein the machine or device is a glucagon pump or other machine or device, worn and maintained outside the body, capable of delivering glucagon from the said pump, machine, or device to the intraosseous space and/or the blood contained within said space and/or the blood contained within the blood vessels within said space, via tubing and/or conductors that extend to said insulin pump or other machine or device.

11. The apparatus of claim 8 wherein the machine or device is a combination of pumps or other machines or devices, worn and maintained outside the body, in contact with the intraosseous space or with blood within the intraosseous space or the blood vessels contained within said space, together with tubing and/or conductors, said machine or device being capable of detecting, monitoring, registering and/or recording the glucose concentration of the blood it contacts and capable also of delivering insulin from the said pump, machine, or device to the intraosseous space and/or the blood contained within said space and/or the blood contained within the blood vessels within said space, the timing and quantity of said delivery to be governed by the blood glucose concentration in accordance with physiologic need for insulin and capable also of delivering glucagon from the said pump, machine, or device to the intraosseous space and/or the blood contained within said space and/or the blood contained within the blood vessels within said space, the timing and quantity of said delivery to be governed by the blood glucose concentration in order to prevent and/or abort an episode of hypoglycemia also known as insulin shock and/or hypoglycemic shock.

12. The apparatus of claim 1, further comprising antimicrobial seals between the tooth supporting bone and the implant.

13. The apparatus of claim 12, wherein the antimicrobial seals comprise dental sealant/cement.

14. The apparatus of claim 3, further comprising piping or tubing that leads from said member through a skin tunnel (like the “piercings” that accommodate jewelry) traversing the mucosa, muscles, subcutaneous tissues and skin situate between (i) the maxillary and mandibular bones (the buccal/“cheek” area) or (ii) the lower labia and mental protuberance (between the “lower lip” and “chin”).

15. The apparatus of claim 1, comprising at least one implant that bears two lumens (“double-barreled”) one lumen equipped with a one way valve that permits inward flow only and one equipped with a one-way valve that permits outward flow only.

16. The apparatus of claim 1, configured for infusion of electrolytic fluid and medication including insulin, “clotting factor(s)” used for treatment of hemophilia, and chemotherapeutic agents used to treat cancer.

17. The apparatus of claim 1, configured for use in connection with at least one of parenteral nutrition or hyperalimentation.

18. The apparatus of claim 1, configured for the withdrawal of blood.

19. The apparatus of claim 1, configured for delivery to/replenishment of/replacement of the hormone glucagon to a reservoir situated within said device, which device, in turn, (a) is implanted beneath the skin, (b) in contact with blood, (c) sensitive to blood glucose concentration, and (d) releases glucagon to the blood should such glucose levels fall too low, thereby avoiding “insulin shock,” and/or “hypoglycemia.”

20. The apparatus of claim 1, further comprising a cap for fitting on said first portion of said implant, said cap being closed by a membrane so as to define a space filled with an antimicrobial substance, and membrane being ruptured by said first portion when said cap is secured to said first portion, to thereby release said antimicrobial substance.

21. The apparatus of claim 20, wherein said first portion of said implant has external threads, and said cap has internal threads that mate with said external threads to secure said cap to said first portion.

22. The apparatus of claim 20, wherein said antimicrobial substance is one of a liquid and a gel.

23. A method for installing the apparatus of claim 1, comprising:
(a) forming a tunnel vertically upward or downward through tooth-supporting bone to intraosseous space;
(b) placing within the tunnel said implant; and
(c) sealing said implant to the bone.

24. A cap comprising;
a housing closed at a first end and having a membrane which defines a closed space within said housing; and
an antimicrobial substance within said closed space, said membrane being capable of being ruptured by a member received within said housing, to which member said cap is releaseably secured, to thereby release said antimicrobial substance.

25. The apparatus of claim 24, wherein said member is a portion of a implant in a living body, said member having external threads, and said cap having internal threads that mate with said external threads to secure said cap to said member.
